(19) United States
(12) Patent Application Publication
(10) Pub. No.: US 2001/0000143 A1
(43) Pub. Date: Apr. 5, 2001

(54) Process for the manufacture of a pulverous preparation

(75) Inventors: Alexander Bausch, Lorrach (DE); Siegfried K. F. Peter, Uttenreuth-Weiher (DE); Kurt Steiner, Starrkirch-Wil (CH); Hansjorg Stoller, Reinach (CH); Eckhard Weidner, Erlangen (DE)

Correspondence Address: Mark E. Waddell, Esq., BRYAN CAVE LLP, 245 Park Avenue, New York, NY, 10167-0034, US

(73) Assignee: ROCHE VITAMINS INC.

(21) Appl. No.: 09/726,876

(22) Filed: Nov. 30, 2000

Related U.S. Application Data
(63) Continuation of application No. 08/953,952, filed on Oct. 10, 1997, now abandoned.

(30) Foreign Application Priority Data
Oct. 14, 1996 (EP) 96116420.9
Nov. 25, 1996 (CH) 2895/96

(51) Int. Cl. B01J 2/00; B01J 2/04; B01J 2/06; B29B 9/00; A61K 9/14

(52) U.S. Cl. 424/489; 424/484; 514/951; 264/5; 264/7; 264/12

(57) ABSTRACT

A process for producing pulverous, i.e., powdered, preparations from sparingly soluble solid materials is disclosed. The process utilizes dimethyl ether under conditions of elevated temperature and pressure to dissolve the solid material. Upon release of the pressure, the solid material precipitates as a fine powder and the gaseous dimethyl ether is released or drawn off.

### BACKGROUND OF THE INVENTION

1. The invention is concerned with a process for the manufacture of a pulverous active substance or pulverous preparation which contains an active substance finely distributed in a matrix component consisting of at least one adjuvant. The active substance is a pharmaceutical, a pharmaceutical precursor, a diagnostic, a fine chemical, a vitamin, or a carotenoid, especially β-carotene. By “pulverous” is meant a finely divided material, such as a powder.

2. Carotenes are hydrocarbons which, on the basis of numerous conjugated double bonds, often have an intensive colour (mainly red to yellow). Together with oxygen-containing compounds (xantophylls) they belong to the carotenoid group. These are present in many natural substances, namely as mixture of different carotenoids, e.g. in algae, fungi, vegetable oils, carrots, paprika. Colour concentrates which are used as physiologically harmless colorants in the cosmetic or foodstuff industry can be obtained from certain natural products. Red carotenoids from paprika are used, for example, for the colouring of lipsticks. Disadvantages of these colour concentrates are the different impurities depending on the starting product and the non-uniform composition of the colour-imparting components.

3. For this reason various syntheses for the production of nature-identical carotenoids especially β-carotenes, have been developed. The preparation of pure carotenes is indispensable primarily with respect to their pharmaceutical application. β-Carotene is of particular interest in this respect. β-Carotene is used as a vitamin A precursor in medicinal preparations. Having regard to its antioxidative activity it is also used in the prophylaxis and therapy of certain cancers. A large number of different synthetic routes for the production of β-carotene have been developed in the past decades. In the industrially used syntheses, e.g. according to Karrer, Pommer, Inhoffen and Isler, the β-carotene obtained in the last step is extracted from the reaction solution with alkanes, e.g. heptane, or chlorinated hydrocarbons, e.g. methylene chloride. After drying there is obtained a solid product which is usually pulverized by milling or dissolved in a conventional solvent, subsequently incorporated into a matrix component and the matrix component containing the thus finely distributed active substance is converted into a powder.

4. A disadvantage is that the solvent concentration in the end product can only be decreased to a value which is harmless to health with the use of considerable technical resources. Furthermore, it is not possible or only very difficult to produce particles having sizes of <10 μm by milling. However, smaller particle sizes are desirable having regard to bioavailability.

5. The object of the present invention is to manufacture a pulverous preparation selected from the groups of pharmaceuticals, pharmaceutical precursors, diagnostics, fine chemicals or carotenoids, especially β-carotene having a particle size of less than 10 μm, especially less than 1 μm.

6. Carotenoids, especially β-carotene and a lot of pharmaceuticals are insoluble in water and have only a very low solubility in most organic solvents. This property and the availability in a relatively large particle size stand in the way of a direct use of β-carotene, for example, for the colouring of aqueous foodstuffs or as feed additives or in the cosmetic field. Attempts have therefore been made in the past, and it is one object of the present invention, to manufacture carotenoids, especially β-carotene, having a particle size below 10 μm, especially below 1 μm.

7. Due to the poor water-solubility of pharmaceuticals the dissolution and consequently the absorption of the drug is critical for the bioavaibility. Therefore very often the drugs were used in micronized form or as a very fine powder with a particle size smaller than 1 μm.

8. A process in which the β-carotene is dissolved in supercritical fluids, preferably carbon dioxide or dinitrogen monoxide, is proposed in DE-OS 29 43 267. Dinitrogen monoxide has somewhat better dissolving capacity for β-carotene than carbon dioxide. Because of the comparatively poor dissolving properties, pressures up to 500 bar are required in order to obtain a β-carotene concentration of 0.01 wt. % in the supercritical gases. The thus-produced supercritical and highly diluted solutions are rapidly depressurized in a suitable depressurization apparatus, e.g., capillaries. The dissolution capacity of the supercritical fluids for carotene is thereby lost and carotene separates in finely divided form. The particle sizes then lie below 1 μm. The separation of these particles from the very large gas stream is achieved by depressurizing the supercritical solution in an aqueous gelatine solution. Thereby, a certain part of the nanometer particles is retained. The gelatine solution, to which is optionally added other adjuvants, e.g., foam preventers, corn starch, glycerine, is dried and subsequently pressed to a solid oral medicament. The process is overall very expensive, since more than 1000 kg of gas are required for the production of 1 kg of pulverous β-carotene.

9. In an alternative process of Chang and Randolph in Precipitation from Microsize Organic Particles from Supercritical Fluids, AICHE Journal, vol 35, No. 11 (1989):1876-1882, it is proposed to dissolve β-carotene in supercritical carbon dioxide at a high pressure and elevated temperature. The solubility of β-carotene in carbon dioxide is strongly dependent on pressure and temperature. It increases with increasing pressure and increasing temperature. Solubility of β-carotene in carbon dioxide at 35° C. and 500 bar is 0.00049 wt. %. The solubility rises to 0.0016 wt. % in the case of an increase of temperature to 55° C. at constant pressure. According to the investigations of Chang and Randolph β-carotene can be crystallized out from the supercritical gas phase by slow pressure and or temperature lowering and the particle sizes, which likewise lie in the nanometer range, and the crystal form can be varied within certain limits by adjusting the speed of the pressure and temperature changes. Disadvantageous in this process are the long dissolution and crystallization times, which lie in the range of 30 min. to several hours. Having regard to the poor solubility, the room-time yields of the process are so low that it is not possible to obtain large amounts of powder in an economical manner.

10. Jay and Steytler in “Nearcritical Fluids as Solvents for β-Carotene”, J. of Supercrit. Fluids, 5 (1992):274-282, investigated the solubility of β-carotene in various supercritical (CO₂, N₂O, C₂H₆, C₂H₄, Xe, SF₆, C₃H₈, CHClF₂, NH₃, CCl₂F₂, SO₂, n-C₄H₁₀) and liquid (n-C₆F₁₄, n-C₅H₁₂, n-C₆H₁₄, n-C₇H₁₆, c-C₆H₁₂, C₂(CH₃)₄, C₆H₆, CCl₄, C₂Cl₄, CS₂, C₂H₅OH, (CH₃)₂CO, CH₂Cl₂) solvents. β-Carotene has only a low solubility in the solvents investigated. The highest concentration of β-carotene in supercritical fluids was measured at 0.035 wt. % for ethylene at 55° C. and 500 bar. Dissolution in liquid sulphur dioxide is 0.59 wt. % at 15° C. The concentration of β-carotene lies below 1 wt. % in the solvents which are liquid at room temperature. An exception is CS₂, in which 3.5 wt. % of β-carotene dissolves at room temperature. The disadvantages of the processes described above, which result, inter alia, from the limited solubility of the β-carotene in dinitrogen monoxide and carbon dioxide, can not be overcome by using the other solvents investigated by Jay and Steytler.

11. A process is proposed in WO 95/21688 in which almost critical or supercritical or generally formulated highly compressible substances are dissolved in organic fluids or form liquid solutions with organic solids which contain in dissolved form the substance to be pulverized. Highly compressible substances which are named are: CO₂, NH₃, N₂O, C₂H₆, C₂H₄, C₃H₈, C₃H₆, CCLF₃, CH₃F, CCl₂F₂, SO₂, n-C₄H₁₀, i-C₄H₁₀, n-C₅H₁₂, as well as at corresponding pressures and temperatures C₂H₅OH, CH₄OH, H₂O, isopropanol, isobutanol, benzene, cyclohexane, cyclohexanol, pyridine, o-xylene. An essential advantage of this mode of operation is that gases have substantially better solubility in organic compounds than vice versa. Typical are values of 5 to 50 wt. % of gas at pressures between 5 and 500 bar, preferably 10 to 200 bar. The gas-containing solutions are rapidly depressurized. The gas is liberated and thereby cools. When a sufficient amount of gas has dissolved out, the cooling can be so strong that the temperature falls below the solidification temperature of the substance to be pulverized. The gas expands strongly during the depressurization. Thereby, the solidified substance disintegrates into very fine particles. In this process between 0.1 kg and 1 kg of gas is required for the production of 1 kg of powder. A further embodiment of this process comprises spraying substances which have a melting point lying above the decomposition temperature. In this case it is proposed to add an adjuvant to the substance to be sprayed. The adjuvant is selected such that the mixture of the substance to be pulverized and the adjuvant has a melting point which lies below the decomposition temperature. The highly compressible component is then dissolved in this liquid mixture or solution. A so-called coprecipitate separates with the rapid depressurizing of the gas-containing solution.

12. In analogy to the procedure described in WO 95/21688 it has been investigated whether β-carotene forms liquid solutions with the mentioned and other highly compressible substances. β-Carotene forms liquid solutions with the gases only at temperatures in the region of its melting point of 180° C. In this temperature region the β-carotene isomerizes or decomposes after a very short time.

### SUMMARY OF THE INVENTION

13. The objectives posed for the manufacture of a pulverous composition from a solid material are thus achieved by a process in which the active substance is dissolved in dimethyl ether under elevated pressure and temperature conditions, the thus-formed solution is flash-decompressed in an expansion apparatus and the pulverous solids formed during the expansion are separated from the dimethyl ether liberated.

### BRIEF DESCRIPTION OF THE FIGURES

14. FIG. 1 shows diagrammatically a simple process sequence based on an apparatus usable for producing the pulverous compositions using dimethyl ether as the solvent;

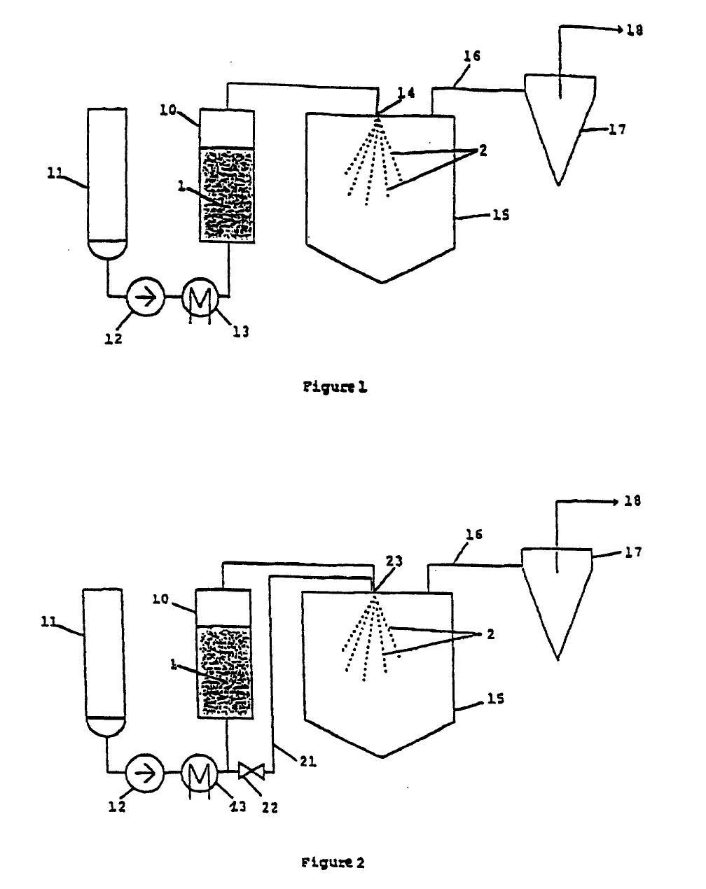

15. FIG. 2 shows diagrammatically a preferred process sequence based on a preferred embodiment of the apparatus according to FIG. 1;

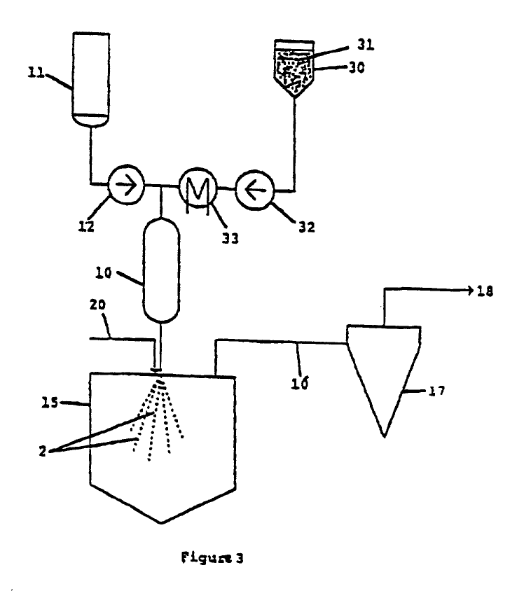

16. FIG. 3 shows diagrammatically a further preferred embodiment of the apparatus according to FIG. 1.

### DETAILED DESCRIPTION OF THE INVENTION

17. It has now been discovered that dimethyl ether is useful as a solvent for β-carotene in processes such as those described above where the material to be produced as a powder is dissolved in a solvent at high temperature and pressure, and then the pressure is reduced whereby the solubility of the dissolved material decreases to the point where it precipitates from the solution as a fine powder. In accordance with the present invention, it has been found that dimethyl ether is completely miscible with β-carotene at temperatures considerably lower than β-carotene's melting point. From PCT Patent Publication WO 96/15133 it is already known that dimethyl ether is an excellent solvent at elevated temperature and pressure conditions. The solubility of β-carotene in liquid dimethyl ether is strongly temperature dependent. About 1.1 wt. % of β-carotene dissolve in liquid dimethyl ether (vapour pressure 4 bar) at 25° C. At 60° C. (vapour pressure 15 bar) already 2.7 wt. % dissolve. At higher pressures (up to 300 bar) the solubility is practically not increased in this temperature region.

18. It has now been discovered that only at temperatures above 100° C. is the solubility of β-carotene in dimethyl ether appreciably pressure dependent. Thus, a homogeneous solution of β-carotene and dimethyl ether exists at 105° C. and pressures above 140 bar. In this pressure and temperature region the two substances are miscible without limitation in any ratio.

19. Thus, the present invention comprises a method for producing a pulverous composition of a solid material which process comprises:

20. a) dissolving the solid material in dimethyl ether under a pressure in the range from about 10×10⁵ Pa to about 500×10⁵ Pa and at a temperature in the range from about 40° C. to about 150° C.,

21. b) reducing the pressure on the thus-formed solution to precipitate the solid material as the pulverous composition and to expand the dimethyl ether into a gas, and

22. c) separating the pulverous composition formed in the expansion from the gaseous dimethyl ether.

23. The preferred solid material for use in accordance with the present invention is a carotenoid, especially β-carotene. However, any compound which is soluble in dimethyl ether under the above-described conditions may be used in accordance with the present invention to obtain pulverous compositions of such a compound.

24. The apparatus used to dissolve the solid material in the dimethyl ether under the specified temperature and pressure conditions is not critical. Any conventional apparatus known in the art for such a process may be used in accordance with the present invention. Further, the apparatus for reducing the pressure to precipitate the solid material as the pulverous composition and to separate the pulverous composition from the dimethyl ether gas is also not critical. Any conventional apparatus known in the art for such a process may be used in accordance with the present invention. In addition to the apparatus disclosed herein, a typical apparatus for practicing the present invention is disclosed in U.S. Pat. No. 4,734,451 issued Mar. 29, 1988.

25. In addition to the carotenoids, other materials, such as pharmaceuticals, may be prepared as pulverous compositions in accordance with the present invention. Some examples of pharmaceuticals include compounds as listed below:

| Therapeutic Category | INN (international nonproprietary name) |
|---|---|
| anxiolytic | Diazepam |
| | Bromazepam |
| antidepressant | Moclobemide |
| anesthetic | Midazolam |
| antiviral | Ganciclovir |
| | Zalcitabine |
| | Nelfinavir mesylate |
| proteinase inhibitor | Saquinavir |
| anti-inflammatory | Naproxen |
| | Tenoxicam |
| | Ketorolac |
| antibacterial | Ceftriaxone |
| | Trimethoprim |
| | Sulfamethoxazol |
| antimalarial | Mefloquine |
| antihypertensive | Cilazapril |
| antiseborrheic | Isotretinoin |
| calcium regulator | Calcitriol |
| lipase inhibitor | Orlistat |
| antiparkinson | Tolcapone |
| antiarthritic | Mycophenolate mofetil |
| antithrombotic | Lamifiban |
| endothelin antagonist | Bosentan |

26. Further advantages, features and details of the invention are described by way of example with reference to the drawing and the following examples in more detail.

27. With reference to FIG. 1, β-Carotene 1 is charged in solid form, preferably having a large surface area, into a suitable high-pressure vessel 10. Dimethyl ether is brought from a reservoir 11 by means of a compressor element 12 to the desired pressure, about 60-500 bar, preferably above 100 bar, preheated in a heat exchanger 13 to about 60-140° C., preferably to 80-140° C., and passed through the β-carotene 1. The β-carotene 1 dissolves in the dimethyl ether. The solution is flash-decompressed using a suitable expansion apparatus 14, e.g., a nozzle, orifice plate, capillary, valve or nozzle/diffuser system. In this simple procedure the final pressure in the expansion is in the order of atmospheric pressure. In a preferred embodiment the expansion device 14 is integrated into a spray tower 15. The dimethyl ether gas released is led off or extracted via a line 16. The pulverous solid β-carotene content present therein is separated from the gas stream in a suitable device 17. For this purpose, conventional process engineering equipment such as, e.g., cyclones, sieves, fine filters or electrostatic precipitators can be used. The gas 18 released can, if desired, be recovered. During the expansion a finely divided, non-agglomerating β-carotene powder 2 is obtained.

28. However, it is preferred to decrease the final pressure only to the extent that the complete miscibility between dimethyl ether and β-carotene is eliminated. The β-carotene then precipitates out at elevated pressure as a solid, finely divided, pulverous product 2. The advantage of this variant is the easy recovery of the dimethyl ether.

29. The particle size of the pulverous β-carotene is below 10 μm, especially below 1 μm. The particle size distribution of the product can be influenced by the selection of the dissolution conditions, the shape of the expansion apparatus 14, in particular the nozzle shape, and other conventional process-engineering measures. In this context, the feed of additional gas during the expansion must be mentioned in particular. This can take place via a separate spray line 20 or in a multi-component expansion apparatus 23 instead of the usual expansion apparatus 14, in particular in a multi-component nozzle. The additional gas used can be dimethyl ether or other gases, preferably liquefied gases, such as carbon dioxide or propane as well as nitrogen. The advantage of using liquefied gases as the additional gas is that intensive cooling usually occurs during their expansion. The supersaturation in the free jet after the expansion may thus be established via the mass stream of the additional gas. FIG. 2 shows the preferred apparatus for adding the additional gas. Compressed dimethyl ether is fed in during the expansion of the β-carotene-containing dimethyl ether using a multi-component nozzle 23 via a bypass line 21 having a suitable controllable shut-off apparatus 22.

30. In a further embodiment of the process, which can be seen in FIG. 3, an adjuvant is added to the carotenoid 1, especially β-carotene, with which, even at low temperatures, it forms a liquid solution or a pumpable suspension. As the adjuvant, use is preferably made of polyethylene glycols of various molecular weights. A mixture 31 of adjuvant and β-carotene is charged into a reservoir tank 30 at temperatures at which the mixture 31 is pumpable. The appropriate temperature and concentration conditions may be varied within wide ranges by suitable choice of the adjuvant. If, for example, polyethylene glycol having a molecular weight of 1500 g/mol is used, the mixtures 31 of polyethylene glycol and β-carotene are pumpable without problem at temperatures from 65° C. to a content of 25 wt. % of β-carotene. If polyethylene glycol having a molecular weight of 4000 g/mol is used, at the same β-carotene concentration a temperature of above 80° C. is necessary to obtain a pumpable mixture 31.

31. The liquid mixture 31 of β-carotene and adjuvant is then continuously fed to a high-pressure vessel 10 by means of a suitable conveying element 32, downstream of which, if appropriate, a heat exchanger 33 is further connected. Simultaneously, a gas or gas mixture is fed to a high-pressure vessel 10 from a reservoir 11 via a compressor element 12. In the pressure vessel 10, in accordance with the thermodynamic conditions in the mixture of adjuvant and β-carotene, β-carotene is dissolved and, after the mixture has flowed through the pressure vessel 10, it is expanded. The subsequent procedure is identical to the above-described embodiments. The solution is expanded as already described in an expansion apparatus 15, β-carotene precipitating out as a solid, finely divided pulverous product 2. In a preferred embodiment the gas used is dimethyl ether. Surprisingly, it has now been found that fine powdering of β-carotene from the mixture of polyethylene glycol and β-carotene is also possible using carbon dioxide or preferably using mixtures of carbon dioxide and dimethyl ether, without the disadvantages previously described in connection with carbon dioxide occurring.

32. Preferrably, as pressure vessel 10, use is made of a mixer-autoclave, preferably a static mixer, since in this case only a very small high-pressure volume is necessary. However, the use of other mixer-autoclaves, such as agitators, shakers, pumped-circulation autoclaves, is also possible.

33. A further embodiment of the invention is the preparation of the solution of the solid material in the dimethyl ether under the conditions described above, and then mixing the resulting solution while still under the pressure with an aqueous adjuvant solution, followed by reducing the pressure on the solution to precipitate the solid material into the aqueous adjuvant solution and to expand the dimethyl ether into a gas which escapes or is drawn off. The aqueous adjuvant solution would contain conventional substances known in the art to be useful for preparing pulverous compositions comprising the solid material. The thus formed aqueous dispersion of the solid material can then be converted into a powder by known methods, such as, in the case of a carotenoid, by the oil dispersion or starch-catch beadlet technologies.

34. An other embodiment of the invention is to provide a process for the manufacture of a pulverous active substance selected from the groups of pharmaceuticals, pharmaceutical precursors, diagnostics, fine chemicals, vitamins or preferably carotenoids, especially β-carotene in which the active substance is finely distributed in a matrix component, especially to give rise to a high availability, preferably bioavailability and/or high colour intensity. Preferably, the particle size of the active substance should lie below 1 μm, especially between about 0.05 μm and about 0.5 μm.

35. Further advantages and feature of the invention will be evident from the following description of embodiments.

36. The process for the manufacture of a pulverous active substance in which the active substance is finely distributed in adjuvant(s) which act as a matrix component, according to the invention is not only simpler, but also more economical than comparable known processes. In particular, the preparation manufactured according to the process in accordance with the invention using dimethyl ether as the compressed gas is distinguished by the advantage that, because of the high dissolution capacity of dimethyl ether for a large number of active substances, it has a high content of active substance which, moreover, is distributed in the matrix component in the form of very small particles and accordingly owing to its large surface has an excellent availability, especially bioavailability and colour intensity, when the preparation is used.

37. Possible adjuvants are at least waxes, fats, hydrocolloids, especially starch or a starch derivative such as, e.g., maltodextrin, gelatines, plant gums such as gum arabic, polysaccharides, sacharose, proteins of animals such as, e.g., lactoprotein, plantprotein and/or proteins of fermentative origin, synthetic polymers such as polyethylene glycol, polyvinylpyrollidone, polylactates or polyacrylates.

38. For example, the above mentioned properties come to full fruition in the case of a preparation in accordance with the invention in which the active substance is a carotenoid, especially β-carotene, and the adjuvant(s) for the matrix component is/are fish gelatine, vegetable proteins or a starch derivative. Thereby, the matrix component acts, inter alia, as a protection for the active substance or for its stabilization and is responsible for an optimal resorption and for a water dispersibility of the final preparation which may by required. The β-carotene particles embedded in the matrix component have a size of 0.05-0.5 μm for an optimal availability, especially bioavailability and/or high colour intensity. The pulverous preparation comprising the adjuvant matrix component and the β-carotene has a preferred size of the individual particles of 50-500 μm depending on the purpose of use.

39. When other active substances, preferably pharmaceuticals as defined above or pharmaceutical precursors, for example retinoids or polyunsaturated fatty acids, are used, it is advantageous when the particle size of the fine active substance embedded in the adjuvant matrix component lies between about 0.05-1 μm, especially about 0.2 μm.

40. Examples 1-6 provide further exemplification of the invention, but are not intended to limit the scope thereof.

### EXAMPLE 1

41. 250 g of solid trans-β-carotene were charged into an autoclave having a volume of 1 l. The autoclave was thermostatted electrically to a temperature of 105° C. 500 g of dimethyl ether (cosmetic grade) were pumped into the autoclave. Dissolution of the carotene took about 60 minutes, with shaking of the autoclave. The pressure was then 175×10⁵ Pa. A sample was taken at both the top and bottom lids of the autoclave. The sample compositions were determined gravimetrically. Both samples gave a carotene content of 33 wt. % and a dimethyl ether content of 67 wt. %, i.e. a homogeneous phase of carotene and dimethyl ether was present in the autoclave.

42. The autoclave was connected to a spray tower via a heated high-pressure line, kept at 105° C., and having an internal diameter of 3 mm. A solid-cone nozzle (Schlick, type V121, bore hole 0.3 mm, angle of spray 30° C.) was fixed at the end of the high-pressure line below the spray-tower lid. The spray procedure was started by opening a shut-off valve between autoclave and expansion apparatus. Through a viewing port in the spray tower the formation of intensive red solid particles was observed immediately. During the spraying process the pressure in the autoclave was maintained by supplementation with fresh dimethyl ether. The gas liberated in the spray tower was fed to a cyclone together with fines portion of the powder. In the cyclone the carotene was virtually quantitatively separated.

43. After completion of the spray procedure the spray tower was opened. 230 g of finely particulate carotene powder were taken off. About a further 20 g of powder were present in the cyclone. The concentration of residual solvent resulting from the industrially employed synthesis process was considerably decreased in comparison with the starting material. The residual content of dimethyl ether was still greatly below that of the methylene chloride.

### EXAMPLE 2

44. 200 g of trans-β-carotene were stirred into 400 g of liquid polyethylene glycol having a molecular weight of 1500 g/mol at a temperature of 75° C. The mixture had roughly the viscosity of honey at this temperature. It was charged in the liquid state into an autoclave having a volume of 1 l. The autoclave was thermostatted electrically to a temperature of 85° C. 350 g of dimethyl ether (cosmetic grade) were pumped into the autoclave. The pressure was 200×10⁵ Pa. After a shaking time of 30 min. the spray procedure as described in Example 1 was started. A commercial two-component nozzle having a bore hole diameter of 0.4 mm was used. The solution of polyethylene glycol/β-carotene and dimethyl ether was passed through the inner nozzle. In the annular gap, during the spray procedure, sufficient additional dimethyl ether was added such that the temperature in the spray tower was 25° C. Through a viewing port in the spray tower the formation of intensive red solid particles was observed. During the spray procedure the pressure in the autoclave was maintained by supplementation with fresh dimethyl ether. The gas liberated in the spray tower was fed to a cyclone together with the solids. In the cyclone the solids were separated off virtually quantitatively as very fine powder.

45. After completion of the spray procedure the spray tower was opened. 550 g of finely divided coprecipitate of polyethylene glycol 1500 and β-carotene were taken off. About a further 112 g of powder were present in the cyclone.

### EXAMPLE 3

46. 167 g of trans-β-carotene were stirred into 500 g of liquid polyethylene glycol having a molecular weight of 4000 g/mol at a temperature of 90° C. The mixture was charged in the liquid state into an autoclave having a volume of 1 l. The autoclave was thermostatted electrically to a temperature of 80° C. 350 g of dimethyl ether (cosmetic grade) were pumped into the autoclave. After a shaking time of 30 min. the spray procedure was started. The spraying was performed in a similar manner to Example 1. The temperature in the spray tower was set at 40° C. Through a viewing port in the spray tower the formation of intensive red solid particles could be observed immediately. During the spray procedure the pressure in the autoclave was maintained by supplementation with fresh dimethyl ether. The gas liberated in the spray tower was fed to a cyclone together with the fines portion of the powder. After completion of the spray procedure the spray tower was opened. 600 g of finely divided coprecipitate of polyethylene glycol 4000 and β-carotene were taken off. About a further 60 g of powder were present in the cyclone.

### EXAMPLE 4

47. 167 g of trans-β-carotene were stirred into 500 g of liquid polyethylene glycol having a molecular weight of 1500 g/mol at a temperature of 60° C. The mixture had roughly the viscosity of thin honey at this temperature. It was charged in the liquid state into an autoclave having a volume of 1 l. The autoclave was thermostatted electrically to a temperature of 60° C. Carbon dioxide was pumped into the autoclave to a pressure of 250×10⁵ Pa. After shaking time of 30 min. the spray procedure was started. A commercial two-component nozzle having a bore hole diameter of 0.4 mm was used. The solution of polyethylene glycol/β-carotene and carbon dioxide was passed through the inner nozzle. In the annular gap, during the spray procedure sufficient additional carbon dioxide was added such that the temperature in the spray tower was 0° C. Through a viewing port in the spray tower the formation of intensive red solid particles could be observed immediately. During the spray procedure the pressure in the autoclave was maintained by supplementation with fresh carbon dioxide. The gas liberated in the spray tower was fed to a cyclone together with the fines portion of the powder. The solid was separated off in the cyclone virtually quantitatively. After completion of the spray procedure the spray tower was opened. 450 g of finely divided coprecipitate of polyethylene glycol 1500 and β-carotene were taken off. About a further 210 g of powder were present in the cyclone.

### EXAMPLE 5

48. 2 kg of carotene were stirred into 10 kg of polyethylene glycol having a molecular weight of 1500 g/mol and a temperature of 70° C. in a reservoir having a volume of about 20 l. The thin solution was brought to a pressure of 150×10⁵ Pa by means of a metering pump having an output of 8 kg/h. The mixture of polyethylene glycol and β-carotene was mixed with dimethyl ether in a static mixer having an internal diameter of 10 mm and a length of 500 mm. The temperature was 50° C. The total mass flow rate was 20 kg/h. After the mixing procedure the expansion was performed in a spray tower in a nozzle having an internal diameter of 0.5 mm and a spray angle of 120°. 11 kg of a finely divided coprecipitate of polyethylene glycol and β-carotene were obtained in the spray tower. About 1 kg of fines portion were collected in a downstream filter.

### EXAMPLE 6

49. 250 g of solid trans-apoester (Ethyl-8′-apo-β-carotene-8′oate) were charged into an autoclave. The autoclave was adjusted to a temperature of 67° C. Dimethyl ether (cosmetic grade) were pumped into the autoclave up to a pressure of 150×10⁵ Pa and equilibrated for 30 minutes. 1003 g of the solution was sprayed into a spray-tower via a nozzle having a bore hole of 0.2 mm. The flow rate was 84 g/min. During the spray procedure the pressure in the autoclave was maintained at 150×10⁵ Pa by supplementation with fresh dimethyl ether. The sample composition was determined gravimetrically. The content of the apoester in the sprayed solution was 10 wt % . The obtained apoester has a particle size of 9 μm and 96 wt % of the apoester was trans apoester.

50. Examples 7-9 further explain the invention according to claim 1 and referring to pharmaceuticals as active substance.

### EXAMPLE 7

51. A comparison of solubility's of a number of pharmaceuticals was performed in the following way:

52. Approximately 3-5 g of the pharmaceutical was slightly compressed in an uniaxial press to avoid the formation of a stable suspension. The so compressed powder was given in a pressure chamber with a sapphire glass (30 ml volume). The temperature of the pressure chamber was controlled by water bath. Then the pressure in the chamber was increased using the corresponding gas and equilibrated for 1-3 hours. After equilibration a defined sample (1.0 ml) was drawn under constant pressure and temperature conditions using a high pressure line with a defined volume. This sample was expanded into a liquid with a good solubility for the respective compound. The sample container was afterwards rinsed with the same liquid to collect the residues of the substance in the sample container.

53. The solubility (G/V) was determined either by HPLC or gravimetrically after removing the liquid.

54. The solubility of pharmaceuticals in liquid carbon dioxide and dimethylether is shown below.

| | solubility | | solubility | |
|---|---|---|---|---|
| pharma- | (CO₂₎ | conditions | (CH₃—O—CH₃) | conditions |
| ceutical | [%(g/V)] | [° C./10⁵ Pa] | [%(g/V)] | [° C./10⁵ Pa] |
| Orlistat | 0.6 | 30/100 | 17.8 | 20/4.5 |
| Isotretionin | 0.3 | 45/200 | 6.0 | 45/200 |
| Sulfameth- | 0.1 | 45/140 | 5.4 | 45/140 |
| oxazol | | | | |
| Saquinavir | <0.1 | 45/200 | >10 | 25/100 |
| Diazepam | 0.15 | 45/200 | >10 | 45/200 |
| Moclo- | 0.35 | 45/200 | 3.7 | 45/200 |
| bemide | | | | |
| Bosentan | <0.1 | 45/200 | 9.0 | 45/200 |

### EXAMPLE 8

55. 100 g of solid Orlistat in a container with two sinter plates was charged into an autoclave having a volume of 6 l. The autoclave was kept at a temperature of 40° C. with a water bath. Then the autoclave was filled with gas up to a pressure of 200×10⁵ Pa and equilibrated for 90 min.

56. The autoclave was connected to a second autoclave via a heated high pressure line, kept at 40° C. This second autoclave has a volume of 4 l. The dissolved Orlistat was sprayed into this second autoclave. Thereby the pressure of the first autoclave was kept constant at 200×10⁵ Pa by pumping in additional gas.

57. The resulting volume weighted particle size distribution is as follows:

| | |
|---|---|
| 10% of the particles | ≦0.40 μm |
| 50% of the particles | ≦1.02 μm |
| 90% of the particles | ≦2.43 μm |

### EXAMPLE 9

58. 100 g of solid Saquinavir in a container with two sinter plates was charged into an autoclave having a volume of 6 l. The autoclave was kept at a temperature of 40° C. with a water bath. Then the autoclave was filled with gas up to a pressure of 200×10⁵ Pa and equilibrated for 90 min.

59. The autoclave was connected to a second autoclave via a heated high pressure line, kept at 40° C. This second autoclave has a volume of 4 l. The dissolved Saquinavir was sprayed into this second autoclave. Thereby the pressure of the first autoclave was kept constant at 200×10⁵ Pa by pumping in additional gas.

60. The resulting volume weighted particle size distribution is as follows:

| | |
|---|---|
| 10% of the particles | ≦0.4 μm |
| 50% of the particles | ≦0.9 μm |
| 90% of the particles | ≦1.8 μm |

61. Example 10 describes a process for the manufacture of a pulverous preparation which contains β-carotene finally distributed in a matrix component according to claim 14.

### EXAMPLE 10

62. 39.7 g of solid apoester containing 97.4% trans-ethyl-8′-apo-β-carotene-8′oate, 10.3 g dl-α-tocopherol and 37.9 g corn oil were charged into an autoclave equipped with a stirrer and having a volume of 1.4 l. 390 g liquid dimethyl ether were added. The pressure was raised to 5×10⁵ Pa. Then nitrogen was pumped into the autoclave up to a pressure of 8×10⁵ Pa.

63. A second autoclave connected by a vent tube and having a volume of 2.4 l was charged with a matrix consisting of 124.1 g gelatin, 49.7 g ascorbylpalmitate sodium salt, 251.7 g sucrose and 474.2 g water. Nitrogen was also pumped into the autoclave up to a pressure of 8×10⁵ Pa.

64. Both autoclaves were adjusted to a temperature of 50° C. and maintained at 50° C. The pressure was raised to 12×10⁵ Pa. Apoester was solubilized completely within 15 minutes under stirring resulting in a dark red solution. This solution containing the dissolved apoester was transferred within 3 minutes through a connecting tube to the second autoclave containing the matrix. During the addition of the apoester solution the matrix was stirred vigorously. After 30 minutes of vigorously stirring dimethylether was slowly evaporated within 35 minutes.

65. After removing the pressure residual dimethylether as well as 170 g of water were removed under vacuum in a thin film evaporator.

66. The solution was sprayed using the known starch catch process. A powder containing 9.1 wt % of apoester was obtained. 94.4 wt % of the apoester was trans apoester. The mean particle size was 0.23 μm.

Claims

1. A process for producing a pulverous composition of a solid material which process comprises:
a) dissolving the solid material in dimethyl ether under a pressure in the range from about 10×10⁵ Pa to about 500×10⁵ Pa and at a temperature in the range from about 40° C. to about 150° C.,
b) reducing the pressure on the thus-formed solution to precipitate the solid material as the pulverous composition and to expand the dimethyl ether into a gas, and
c) separating the pulverous composition formed in the expansion from the gaseous dimethyl ether.

2. The process of claim 1 wherein the solid material is a carotenoid.

3. The process of claim 2 wherein the carotenoid is β-carotene.

4. The process of claim 3 wherein the pressure is in the range from about 60×10⁵ Pa to about 200×10⁵ Pa.

5. The process of claim 4 wherein the temperature is in the range from about 80° C. to about 150° C.

6. The process of claim 5 wherein the temperature is in the range from about 100° C. to about 150° C.

7. The process of claim 4 wherein the pressure is in the range from about 100×10⁵ Pa to about 200×10⁵ Pa.

8. The process of claim 7 wherein the temperature is in the range from about 80° C. to about 150° C.

9. The process of claim 8 wherein the temperature is in the range from about 100° C. to about 150° C.

10. The process of claim 1 wherein that the solid material is a pharmaceutical.

11. The process of claim 10 wherein that the pharmaceutical is diazepam, bromazepam, moclobemide, midazolam, ganciclovir, zalcitabine, nelfinavir mesylate, saquinavir, naproxen, tenoxicam, ketorolac, ceftriaxone, sulfamethoxazole, trimethoprim, mefloquine, cilazapril, isotretinoin, calcitriol, orlistat, tolcapone, mycophenolate mofetil, lamifiban or bosentan.

12. The process of claim 1 wherein a dispersion, which comprises the solid material dispersed in a liquid adjuvant, is dissolved in the dimethyl ether.

13. The process of claim 12 wherein the adjuvant is polyethylene glycol.

14. The process of claim 13 wherein the solid material is a carotenoid.

15. The process of claim 14 wherein the carotenoid is β-carotene.

16. The process of claim 15 wherein the dispersion comprises 1-75 wt. % of β-carotene.

17. The process of claim 16 wherein the dispersion comprises 5-50 wt % of β-carotene.

18. The process of claim 17 wherein the dispersion comprises 10-30 wt % of β-carotene.

19. A process for producing a pulverous preparation of a solid material dispersed in a matrix component comprising an adjuvant, which process comprises:
a) dissolving the active substance under elevated temperature and pressure conditions in a compressed gas in the subcritical or supercritical state,
b) dispersing the solution obtained from a) in an aqueous solution of the adjuvant,
c) removing the compressed gas from the dispersion obtained from b) to precipitate the solid material into the aqueous adjuvant solution,
d) converting the aqueous adjuvant solution into a pulverous preparation.

20. The process of claim 19 wherein the gas is dimethyl ether, the pressure is in the range from about 10×10⁵ Pa to about 1000×10⁵ Pa, and the temperature is in the range from about 50° C. to about 200° C.

21. The process of claim 20 wherein the compressed gas is removed from the dispersion by evaporation.

22. The process of claim 21 the aqueous adjuvant solution is converted into the pulverous preparation by spray drying.

23. The process of claim 22 wherein the solid material is a carotenoid.

24. The process of claim 23 wherein the temperature is in the range from about 50° C. to about 150° C. and the solid material is β-carotene.

25. The process of claim 24 wherein that the adjuvant comprises waxes, fats, hydrocolloids, gelatines, plant gums, polysaccharides, proteins of animal, plant or fermentative origin, polyethylene glycols, polyvinylpyrollidone, polylactates or polyacrylates.

26. The process of claim 25 wherein the adjuvant comprises fish gelatine, plant proteins or a starch derivative.

27. The process of claim 26 wherein the pressure is in the range from about 50×10⁵ Pa to about 500×10⁵ Pa.

28. A pulverous preparation comprising a matrix component in which particles of a solid material are dispersed, said matrix component comprising an adjuvant, and the size of said particles of solid material are in the range from about 0.01 μm to about 3.0 μm.

29. The pulverous preparation of claim 28 wherein the size of said particles of solid material are in the range from about 0.05 μm to about 0.5 μm.

30. The pulverous preparation of claim 29 wherein the solid material is a carotenoid and the adjuvant is fish gelatine, a plant protein or a starch derivative.

31. The pulverous preparation of claim 30 wherein the solid material is β-carotene.
